(19) United States
(12) Patent Application Publication
(10) Pub. No.: US 2023/0157945 A1
(43) Pub. Date: May 25, 2023

(54) Nasal Hygiene Method and Formulation

(71) Applicant: ACTUAL NATURAL HEALTH & WELLNESS PRODUCTS, INC., ASHTON, MD (US)

(72) Inventors: Paul A. Lobel, Ashton, MD (US); Bruce Artman, Eldersburg, MD (US)

(21) Appl. No.: 18/101,047

(22) Filed: Jan. 24, 2023

Related U.S. Application Data
(63) Continuation-in-part of application No. 17/464,122, filed on Sep. 1, 2021.
Application No. 17/464,122 is a continuation of application No. 16/597,729, filed on Oct. 9, 2019, now Pat. No. 11,110,056.
Application No. 16/597,729 is a continuation-in-part of application No. 16/403,262, filed on May 3, 2019, now Pat. No. 10,945,982.
(60) Provisional application No. 62/743,328, filed on Oct. 9, 2018.
(60) Provisional application No. 62/666,561, filed on May 3, 2018.
(60) Provisional application No. 63/302,453, filed on Jan. 24, 2022.

(51) Int. Cl. A61K 9/00; A61K 33/00; A61K 47/02; A61K 31/05; A61K 31/202; A61K 31/201

(52) U.S. Cl. CPC A61K 9/0043; A61K 33/00; A61K 47/02; A61K 31/05; A61K 31/202; A61K 31/201

(57) ABSTRACT

A composition for and method of practicing nasal hygiene to wash and moisturize the nasal passage. Where the nasal wash comprises a liquid homogenized compound of water, chlorine dioxide (or source thereof), olfactory stimulants, unsaturated fatty acids, and fixing agents. Where the unsaturated fatty acid component comprises from 0.01% to 1% by weight of the compound.

This application is a continuation in part of co-pending U.S. patent application Ser. No. 17/464,122, filed Sep. 1, 2021, which is a continuation of U.S. patent application Ser. No. 16/597,729, filed Oct. 9, 2019, now U.S. Pat. No. 11,110,056, issued on Sep. 7, 2021, which is a continuation-in-part of U.S. patent application Ser. No. 16/403,262, filed May 3, 2019, now U.S. Pat. No. 10,945,982, issued on Mar. 16, 2021, which claims priority pursuant to 35 U.S.C. 119(e) to U.S. Provisional Patent Application Serial No. 62/743,328 filed Oct. 9, 2018, and U.S. Provisional Patent Application Ser. No. 62/666,561 filed May 3, 2018. This application also claims priority pursuant to 35 U.S.C. 119(e) to co-pending U.S. Provisional Patent Application Ser. No. 63/302,453 filed Jan. 24, 2022. The entire disclosure of these six applications is incorporated herein by reference.

### FIELD OF THE INVENTION

This invention relates to a system and method for rinsing a human nasal passage.

### BACKGROUND OF THE INVENTION

The nose is a vital organ, but is often ignored. The nose is responsible for filtering, warming, and humidifying the air one breathes. Increased air pollution overwhelms the natural function of the nose. Air pollution, including ozone, nitric oxide, and nitrogen dioxide can cause lung injury if they are not filtered by the nose. The condition of dry nose reduces the natural filtering and humidifying function of the nose. Air pollution can cause irritation of nose.

In the United States, nasal hygiene is rarely practiced as a daily or even frequent periodic routine. Daily nasal hygiene is a known method to help clean the nasal passages. However, none of the known solutions for nasal hygiene provide moisturizing compounds and antioxidant compounds. None of the known solutions for nasal hygiene provide pain relief or reduce irritation of the nose and nasal passages.

Plain water, the universal cleanser, turns out to be a poor choice for nasal hygiene. As pointed out by Douglas Hoffman MD, while salt solutions containing higher concentrations than body tissues (so-called hypertonic solutions) draw water out of the tissues, plain water has the opposite effect, entering the tissues and adding to the swelling pressure. Saline solutions, on the other hand, need careful regulation to avoid levels of salt that irritate healthy and particularly inflamed tissues.

In ear-nose-throat clinics, patients have had noses rinsed with dilute potassium permanganate solutions for disinfecting purposes. This procedure required using large volumes of rinse solution, and users underwent the inconvenience of catching and disposing of the spent solution after use.

Cannabinoids are the various chemical constituents of hemp or cannabis. The known cannabinoids include, CBD (Cannabidiol), CBG (Cannabigerol), CBC (Cannabichromene), CBDA (Cannabidiolic acid synthase) CBDV (Cannbidivarin) CBN (Cannabinol), Delta-9-Tetrahydrocannabinol (Δ9 THC), Delta-8-Tetrahydrocannabinol (Δ8 THC), THCA (Tetrahydrocannabinolic acid), THCV (Tetrahydrocannabivarin), or any of the other 115 known cannabinoids and unknown cannabinoids. Cannabis is widely known to be consumed via the mouth, either by smoking or eating foods containing cannabis. Use of cannabis via the nasal passages is not known and would be painful due to the sensitive nature of the mucus membranes in the nasal passages.

### SUMMARY OF THE INVENTION

In accordance with this invention, there is provided an improved system and method of practicing nasal hygiene using compositions containing cannabinoids.

The inventors have discovered that concentrations of cannabinoids as low as 0.001% by weight when included in a nasal spray were found to provide effective nasal hygiene, pain relief, and a reduction in inflammation. Concentrations of cannabinoids as high as 5% by weight provide effective nasal hygiene without negative effects on the homogenization of the nasal spray.

A homogenized mixture of water, cannabinoid, an olfactory stimulant, and inorganic salt when applied to a nasal passage as a mist delivering 0.08 mL to 0.18 mL in each nostril is effective as a nasal wash. A solubilizing agent particularly suited for cannabinoids may also be added, such as those disclosed in U.S. patent application Ser. No. 17/527,465, filed Nov. 16, 2021.

It is believed that cannabinoids in combination with chlorine dioxide are particularly useful in providing effective nasal hygiene, pain relief, and reducing inflammation of the nose and nasal passages.

The system of rinsing a human nasal passage using a mixture comprising water, cannabinoid, a source of chlorine dioxide, and olfactory stimulant dispensed using an apparatus into the nasal passage as a mist is particularly effective at cleaning the nasal passages and reducing inflammation of the nose. It has also been found the addition of one or more of the following allows for a more reliable system for rinsing the nasal passage: inorganic salt, fixative compounds, solubilizers.

The inventors have discovered that many cannabis sativa plant extracts cannot effectively be used for nasal hygiene. The levels of terpene and other naturally occurring compounds in many cannabis sativa plant extracts that are easily tolerated by users of oral and oral vapor delivery devices create irritation of the membranes in the nasal cavity. The membranes in the nasal cavity range from 10-15 μm thick as compared to the healthy epithelium having a mean thickness of 335.59±150.73 μm. According to its location within the oral cavity, the epithelium showed highest values in the region of the buccal mucosa (659.79 μm) and the thinnest one was observed in the mouth's floor (100.07 μm).

As a result, the use of cannabis tinctures as a component of a nasal spray by administration through the nasal passages is discouraged due to the negative side effects caused by terpenes and other naturally found compounds in the full-spectrum and broad-spectrum cannabinoids. The invention eliminates the inclusion of indiscriminate terpenes to a non-detectable level by a user.

It was found that prior art nasal sprays including cannabis extracts employ a non-water-soluble oil extract. It has been found that this non-water-soluble oil causes a pattern of inconsistent dosage when attempting to introduce CBD (Cannabidiol) into the nasal cavity due to the cannabidiol separating while static, therefore must be shaken to mix the ingredients. The recommended amount of or length of or vigorousness of the shaking requirement is inconsistent due to its nature therefore the amount of CBD (Cannabidiol) in each dose cannot be determined thus yielding in inconsistent results.

There is also provided, in accordance with this invention, a novel non-irritating hygienic composition for use in the system consisting essentially of a product of compounding under homogenizing conditions water, 0.01% to 3% by weight of at least one olfactory stimulant; 0.001% to 5% by weight of cannabinoid; 0 to 5% by weight of at least one fixative compound less volatile than the olfactory stimulant; 0.1 to 2.5% by weight of at least one inorganic salt selected from the group consisting of alkali metal chloride, alkali metal bicarbonate, and alkali metal chlorate.

There is further provided, in accordance with this invention, a novel non-irritating hygienic mixture for use in the system consisting essentially of a product of compounding under homogenizing conditions water, 0.01% to 5% by weight, as chlorine dioxide, or a source of chlorine dioxide; 0.01% to 3% by weight of at least one olfactory stimulant; 0.001% to 5% by weight of cannabinoid; 0 to 5% by weight of at least one fixative compound less volatile than the olfactory stimulant; 0.1 to 2.5% by weight of at least one inorganic salt selected from the group consisting of alkali metal chloride, alkali metal bicarbonate, and alkali metal chlorate.

It has been found that the ingredients of the composition interact cooperatively to provide an enhanced beneficial effect to the user beyond the individual effect of each individual ingredient. The inclusion of cannabinoid in particular enhances the effect of the other ingredients. The nature of the interaction is not fully understood. It is believed that the combination of chlorine dioxide with the cannabinoid functions in the composition of the invention by activating and enhancing the effectiveness of one or more other components of the composition.

Further, the use of at least three olfactory stimulants has been found to be more effective than a single olfactory stimulant.

The further addition of at least one unsaturated fatty acid to the mixture in the amount of 0.01% to 1% by weight by weight increases the moisturizing effect of the composition on the nasal passages and reduces oxidizing air pollution. The addition of at least one unsaturated fatty acid also enhances the ability of the nasal passages to filter particulate matter from polluted air. Preferably, the unsaturated fatty acid is a monounsaturated fatty acid, such as omega-7 fatty acids and omega-9 fatty acids, or a polyunsaturated fatty acid, such as omega-3 fatty acids. The compound may also contain combinations of monounsaturated fatty acid and polyunsaturated fatty acid. Preferably, the composition comprises plant-based sources of unsaturated fatty acid.

The term “consisting essentially of” is used in its art-recognized meaning to indicate that the composition is open only to such additional ingredients as do not adversely affect its beneficial properties. In particular, such ingredients, and concentrations of ingredients, as render the composition irritating to the mucous membrane inside the nose, are excluded. As an example of an additional ingredient that does not adversely affect the beneficial properties and can enhance the benefit of the composition, a fixative compound less volatile than the olfactory stimulant is given.

The term “product compounded under homogenizing conditions” defines a product that has been subjected to heat treatment and mechanical action sufficient to minimize a tendency to separate into two liquid phases. Heat treatment at moderately elevated temperatures in the range of 30 to 55° C. for a period of 1 to 2000 seconds is generally sufficient. Equipment for applying mechanical action is well known in the art and commercially available. The inventors have discovered the disclosed product remains homologous for at least 90 days and inclusion of an emulsifier with the cannabinoid provides a mixture that remains homogonous for at least 180 days.

Practicing nasal hygiene by the use of the method of applying to a person's nostril a mist of nasal wash composition comprising the product of compounding under homogenizing conditions water, an amount of a source of chlorine dioxide, 0.1 to 1% by weight cannabinoid; 0.1 to 3% by weight of olfactory stimulant; and 0 to 5% by weight of at least one fixative compound; and blowing air out of the nostril. and composition of the invention, nasal irritation is reduced. Daily flushing or washing out the nose according to the invention has been found through testing to reduce inflammation, reduce pain, and moisturize the nasal passages more effectively than previous washes.

### DESCRIPTION OF THE PREFERRED EMBODIMENTS

The system and method of the inventions uses a mixture or composition that includes at least one cannabinoid. In particular, the cannabinoids: CBG (Cannabigerol), CBC (Cannabichromene), CBDA (Cannabidiolic acid synthase), CBDV (Cannbidivarin), Delta-8-tetrahydrocannabinol an isomer of delta-9-tetrahydrocannabinol (THC), CBN (Cannabinol), Delta-9-Tetrahydrocannabinol (THC), THCA (Tetrahydrocannabinolic acid), THCV (Tetrahydrocannabivarin), and CBD (Cannabidiol) are preferred.

In a preferred embodiment at least two cannabinoids are combined. Some embodiments also include cannabinoids (or combinations of cannabinoids) that are water soluble or water dispersible. Preferred embodiments include a combination of at least five cannabinoids that are water soluble or water dispersible to provide a full or broad spectrum of various cannabinoids. It is further preferred that the cannabinoids used in the composition are converted to isolate. The use of cannabinoid isolate removes most of the terpenes found in cannabis. It is preferable that the mixture or composition explicitly excludes terpenes found in traditional extractions of hemp because these are particularly irritating to the nose and nasal passages.

Some of the preferred embodiments of the invention include a source of chlorine dioxide. The inclusion of chlorine dioxide in nasal spray is described in U.S. Pat. No. 7,029,705 and is hereby incorporated by reference. The preferred embodiment includes sufficient chlorine dioxide source to provide 0.01 to 5% by weight chlorine dioxide upon conversion thereto. More preferably, 0.1 to 2% chlorine dioxide.

In one embodiment of the invention, only water-soluble isolate versions of cannabinoids are used as a component of the spray. Testing has show that use of water-soluble isolate prevents irritation, including watery eyes, ringing in the ears and lengthy discomfort in the nose.

In the preferred embodiments, the cannabinoids incorporated into the nasal spray are 100% water dispersible and combined with a natural emulsifier guaranteeing the cannabinoids maintain solubility providing consistent dosage in every dose. Examples of such water-soluble cannabis formulations include those disclosed in U.S. Pat. Nos. 9,907,823, 10,046,018, and 10,328,111 and U.S. patent application Ser. No. 16/977,200.

In accordance with another invention, there is provided an improved method of practicing nasal hygiene comprising the steps of applying to a person's nostril a non-irritating hygienic composition consisting essentially of a product of compounding under homogenizing conditions water, 0.01% to 5% by weight, as chlorine dioxide, of a source of chlorine dioxide; 0.01% to 3% by weight of at least one olfactory stimulant; 0.001% to 5% by weight of cannabinoids; 0 to 5% by weight of at least one fixative compound less volatile than the olfactory stimulant 0.1 to 2.5% by weight of at least one inorganic salt selected from the group consisting of alkali metal chloride, alkali metal bicarbonate, and alkali metal chlorate; and 0.0002 to 0.006% (as aluminum) by weight of at least one water soluble aluminum compound, provided that the total concentration of inorganic salt is in the range from 0.6% by weight to 2.5% by weight, holding the composition within the nostril for a hygienic holding period, and discharging the composition from the treated nostril or gently inhaling the composition of Cannabinoids to achieve the desired absorption to achieve maximum bio-availability into the mucus membrane.

The olfactory stimulant ingredient can be a cycloaliphatic alcohol, a cycloaliphatic ketone, an aromaticnon-phenolic hydroxyl compound, an aromatic ether, a phenol having nine or more carbon atoms, or a mixture of such compounds. The olfactory stimulant ingredient can also be a mixture of volatile oils or ethereal oils derived from plant materials.

Illustrative cycloaliphatic alcohols include cyclododecanol, 3,3,5-trimethylcyclohexanol, and 4-t-butylcyclohexanol. Illustrative cycloaliphatic ketones include exaltone, fenchone, isophorone, muscone, and 3,3,5-trimethylcyclohexanone. Illustrative aromatic non-phenolic hydroxyl compounds include 1-phenylethanol, 2-phenylethanol, and 1-phenoxy-2-propanol. Illustrative aromatic ethers include diphenyl ether and 2-methoxynaphthalene. Illustrative phenols include p-t-octylphenol and 2,6-di-t-butyl-4-methylphenol.

Particularly preferred olfactory stimulant ingredients include thymol, eucalyptol, borneol, menthol, camphor, oil of eucalyptus, pine oil, and gum benzoin. Fixative compound when present is less volatile than the olfactory stimulant ingredient and can be, for example, an essential oil. Particularly preferred fixative compounds include oil of sweet birch, oil of spearmint, oil of pine, and cinnamon.

Certain oils extracted from plant materials include olfactory stimulants compounds as well as less volatile fixative type compounds and can serve as sources of both. Particularly preferred dual function oils of this type include basil, bergamot, lemon, citrus, jasmine, lemongrass, rosemary, sage, thyme, and vanilla.

The quantity of olfactory stimulant material in the composition of the invention is in the range from 0.01% to 3% by weight, preferably in the range from 0.05% to 1% by weight. The preferred embodiment includes at least three olfactory stimulants. The quantity of fixative material when present is in the range from 0.01% to 5% by weight, preferably in the range from 0.05% to 1% by weight.

The unsaturated fatty acid ingredient can be a monounsaturated fatty acid, such as omega-7 fatty acids and omega-9 fatty acids, or a polyunsaturated fatty acid, such as omega-3 fatty acids. The unsaturated fatty acid ingredient can also be a mixture of monounsaturated and polyunsaturated fatty acids. Preferably, the unsaturated fatty acids are derived from plant materials.

Illustrative unsaturated fatty acids include palmitoleic acid, vaccenic acid, paullinic acid, oleic acid, erucic acid, elaidic acid, gondoic acid, mead acid, nervonic acid, ximenic acid, hexadecatrienoic acid, a-linolenic acid, stearidonic acid, eicosatrienoic acid, eicosatetraenoic acid, eicosapentaenoic acid, heneicosapentaenoic acid, docosapentaenoic acid, clupanodonic acid, docosahexaenoic acid, tetracosapentaenoic acid, tetracosahexaenoic acid, linoleic acid, gamma-linolenic acid, calendic acid, eicosadienoic acid, dihomo-gamma-linolenic acid, arachidonic acid, docosadienoic acid, adrenic acid, osbond acid, tetracosatetraenoic acid, and tetracosapentaenoic acid.

Particularly preferred unsaturated fatty acid ingredients include walnut oil, clary sage seed oil, algal oil, flaxseed oil, pomegranate seed oil, borage seed oil, and sea buckthorn oil. These oils may also operate as a fixative compound.

The quantity of unsaturated fatty acid in the composition of the invention is in the range from 0.01% to 1% by weight, preferably in the range from 0.05% to 1% by weight.

Preferred cannabinoids include CBD (Cannabidiol), CBG (Cannabigerol), CBC (Cannabichromene), CBDA (Cannabidiolic acid synthase) CBDV (Cannbidivarin) CBN (Cannabinol), Delta-9-Tetrahydrocannabinol (Δ9 THC), Delta-8-Tetrahydrocannabinol (Δ8 THC), THCA (Tetrahydrocannabinolic acid), THCV (Tetrahydrocannabivarin). The total concentration of cannabinoids is 0.001% to 5% by weight. It is best incorporated into the composition as an oil or aqueous extract of hemp. Preferably the concentration of psychoactive Delta-9-Tetrahydrocannabinol is 3% by weight or less. Most preferably the concentration of Delta-9-Tetrahydrocannabinol is between 0.001% and 0.3% by weight.

Various combinations of cannabinoids have been found to be particularly useful in reducing inflammation as part of the disclosed composition or mixture. Cannabidiol (CBD) in combination with Delta-8-Tetrahydrocannabinol (48 THC) is effective at reducing inflammation. In particular, 1% to 4% of CBD combined with 0.04% to 0.08% 48 THC was found to be particularly effective at washing nasal passages and reducing inflammation. Concentrations of CBD at 0.3% by weight were found to provide effective pain relief during testing.

The inorganic salt ingredients of the composition, i.e. alkali metal chloride, alkali metal bicarbonate, and alkali metal chlorate, can be lithium salts, potassium salts, sodium salts, and mixtures thereof. When salts of different metals are used together, for example a sodium salt and a potassium salt, ionization in the solution can associate a metal ion with the anion of a different salt from that supplied with the metal. Thus, a solution in which potassium bicarbonate and sodium chloride are dissolved contains potassium chloride as well as potassium bicarbonate, and sodium bicarbonate as well as sodium chloride.

Without intending to be bound by any theory, it is believed that the inorganic salt ingredient of the composition provides a multiplicity of functions, including adjusting the osmotic strength and the pH of the composition, and limiting the amount of water passing into the mucous membrane while enhancing the cleansing effect. It is therefore preferred to use a combination of two or more inorganic salts according to the invention.

The quantity of each inorganic salt in the composition of the invention is in the range from 0.1% to 2.5% by weight, preferably from 0.3% to 2% by weight, provided that the combined quantity of inorganic salts in the composition of the invention is in the range from 0.6% to 2.5% by weight.

In one preferred embodiment, the inorganic salt sodium chloride is avoided and other inorganic salts, including magnesium chloride, calcium carbonate, and potassium phosphate are used. It is preferable to keep the concentration of sodium chloride below 0.2% by weight.

Optional adjuvants that can be included in low concentrations, typically less than 0.5%, in the composition of the invention to impart desired characteristics include colorants illustrated by sodium copper chlorophyllin, surface active materials illustrated by Polysorbate 80 (an ethoxylated sorbitan ester), and chelating agents illustrated by sodium citrate.

It is a feature of the invention that the practice of nasal hygiene according to the invention requires only moderate and convenient quantities of the composition. It is sufficient for the user to place a convenient quantity of the composition, which can range from about 0.3 to 3 milliliters, into each nostril and retain it for a holding period which can range from a fraction of a minute to about ten minutes or as long as comfortable, and then remove it as by blowing the nose. Any suitable applicator can be used to place the composition inside the user's nose. The preferred method of application is to insert the atomizer tip into the nasal entry point a quarter of an inch but no more than half an inch.

The preferred embodiments use a bottle of 50 mL or less to hold the composition or mixture for application. Preferably the bottle is topped by a manual atomizer pump applicator. Preferably the applicator delivers the composition or mixture as a fine mist. More preferably the manual atomizer pump dispenses 0.08 mL to 0.18 mL of total volume per pump. And, the preferred method dispenses one to two pumps per nostril.

1. A system for rinsing a human nasal passage, the system comprising:
a mixture comprising water, a source of chlorine dioxide, an olfactory stimulant, and a cannabinoid; and
an apparatus for dispensing the mixture into a nasal passage as a mist.

2. The systems of claim 1, wherein said mixture further comprises a second cannabinoid and wherein the total concentration of all cannabinoids is in the range of 0.001% to 5% by weight.

3. The systems of claim 1, wherein the cannabinoid in the mixture comprises delta-8-tetrahydrocannabinol.

4. The system of claim 1, wherein the cannabinoid is delta-9-tetrahydrocannabinol.

5. The system of claim 1 wherein the mixture comprises at least five cannabinoids

6. A method of practicing nasal hygiene comprising the steps of applying to a person's nostril a mist of liquid nasal wash composition comprising the product of compounding under homogenizing conditions water, an amount of a source of chlorine dioxide, 0.1 to 1% by weight cannabinoid; 0.1 to 3% by weight of olfactory stimulant; and 0 to 5% by weight of at least one fixative compound; and blowing air out of the nostril.

7. A system for rinsing a human nasal passage, the system comprising:
a mixture comprising water, olfactory stimulant, inorganic salt, and cannabinoid; and
an apparatus for dispensing the mixture into a nasal passage as a mist and delivering 0.08 mL to 0.18 mL per each activation of the apparatus to the nose.

8. The system of claim 7, wherein the cannabinoid is a water-soluble isolate.

9. The system of claim 7, wherein the mixture is homogonous.

10. The system of claim 10, wherein the mixture remains homogonous for at least 90 days.

11. The system of claim 10, wherein the mixture remains homogonous for at least 180 days.
